(19) United States
(12) Patent Application Publication
(10) Pub. No.: US 2016/0102364 A1
(43) Pub. Date: Apr. 14, 2016

(54) Neuroglioma Molecular Subtyping Gene Group and Use Thereof

(71) Applicant: BEIJING NORMAL UNIVERSITY, Beijing (CN)

(72) Inventors: Xiaolong FAN, Beijing (CN); Yingyu SUN, Beijing (CN); Tao JIANG, Beijing (CN)

(21) Appl. No.: 14/894,197

(22) PCT Filed: May 27, 2014
(86) PCT No.: PCT/CN2014/000535

§ 371 Date: Nov. 25, 2015

(30) Foreign Application Priority Data
May 28, 2013 (CN) 201310202569.1

(51) Int. Cl. C12Q 1/68

(52) U.S. Cl. CPC C12Q 1/6886; C12Q 2600/158; C12Q 2600/118

(57) ABSTRACT

A set of gene groups for predicting or for the auxiliary prediction of the survival time prognosis of patients with neuroglioma consists of a PM gene group and an EM gene group with a total of 68 genes. A subtype marking gene group consisting of two major gene groups co-expressed with PDGFRA and EGFR can stably divide neuroglioma samples from different database sources into three specific subtypes, and can be applied to clinical diagnosis for neuroglioma and judging the survival time prognosis of patients with neuroglioma.

### TECHNICAL FIELD

The present invention relates to the biotechnology field, and particularly to neuroglioma molecular subtyping gene group and use thereof, which mainly involves study of expression profiles of tumor related genes in functional genomics and screening and verification of marking gene groups, that is, screening neuroglioma subtype marking gene groups within human functional genome, and diagnosing subtype of a neuroglioma sample based on the expression level of mRNAs of these genes; and allows for predicting prognosis of a patient.

### BACKGROUND

Neuroglioma is a primary tumor that is most common in central nervous system, and is a major threat to human health. Most patients with high-grade malignant gliomas experience a survival time of only 1-2 years. A low-grade malignant glioma will finally progress into high-grade, but the speed thereof is significantly different in one another individual. Currently, it is still unclear the nature of the variance in progression speed of a low-grade malignant glioma, and no target effective for treating neuroglioma can be found until now.

Diagnosis of a disease is an essential basis for study and development of a therapeutic regime. At present, diagnosis of neuroglioma is still based on morphology. However, due to morphological heterogeneities in glioma and subjective differences from physician in diagnosis, the diagnostic inconsistencies may be up to 30%-40%, and a considerable partion of neuroglioma cannot be definitely diagnosed with morphology. Moreover, the morphology-based diagnosis cannot precisely represent the nature of a disease. Thus, there is a huge constraint in the existing criteria of morphological diagnosis for the research of clinical treatment of gliomas. Recently, a subtyping method based on gene expression opens up a new way to the diagnosis of gliomas. A molecular subtyping method makes it possible to reveal the cytological and genetic nature in the development and progression of gliomas, and can dramatically promote the study of targeted treatment of gliomas. However, there is still a lack of regimes effective for clinically diagnosing neuroglioma based on gene expression so far in worldwide.

### DISCLOSURE OF INVENTION

The present invention has a goal to provide a kit for predicting or auxiliary prediction of survival time prognosis of a patient with neuroglioma.

The present invention provides a kit comprising an agent for measuring expression levels of various genes of a PM gene group and an EM gene group in an ex vivo sample from a patient with neuroglioma to be identified, and a comparison card;

the PM gene group consisting of following 39 genes:

C10orf18, C1QL1, C1orf106, C9orf140, CACNG4, CHD7, CSNK1E, EIF4EBP2, ETV1, FAM5C, KLRC3, LIX1L, LOC283174, LPHN3, LPPR1, MARCKS, MEX3A, MMP16, MYT1, NAV1, NLGN1, NOVA1, NXPH1, OLIG1, OLIG2, PATZ1, PCGF2, PDGFRA, POLR2F, RFX7, SOX4, SOX6, SOX8, TACC2, TMCC1, TSHZ1, ZEB1, ZNF22, ZNF462;

the EM gene group consisting of following 29 genes:

### ACSS3, CDKN2C, DENND2A, DMRTA2, EGFR, ELOVL2, HS3ST3B1, ITGB8, LFNG, NCOA3, NES, NFIA, PDGFA, PMS2P11, POU3F2, PRPF31, RNF180, SALL1, SEC61G, SEMA6D, SHOX2, SNX5, SOCS2, SOX9, TNFRSF19, TRIOBP, UHRF1, VAV3, ZNF558; and

the comparison card recording that:

if the ex vivo sample from the neuroglioma patient to be identified has an average expression level of the PM gene group greater than or equal to that of the EM gene group, the neuroglioma patient to be identified has a survival time prognosis over or optionally over 1.9 years; and

if the ex vivo sample from the neuroglioma patient to be identified has an average expression level of the PM gene group less than that of the EM gene group, the neuroglioma patient to be identified has a survival time prognosis of no more than or optionally no more than 1.9 years.

In above kit, the comparison card may record that:

if the ex vivo sample from the neuroglioma patient to be identified has an average expression level of the PM gene group greater than that of EM gene group, the neuroglioma patient to be identified has a survival time prognosis of, or optionally of, 3.7-4.9 years (for Occidental, particularly for American REMBRANDT Neuroglioma Large Database) or 2.3-2.7 years (for Asian, particularly for neuroglioma cases treated by Beijing Tian Tan Hospital in 2006-2009, the Chinese Glioma Genome Atlas, http://jzl.dajiankang.com/portal.php);

if the average expression level of the PM gene group is equal to that of the EM gene group, the neuroglioma patient to be identified has a survival time prognosis of, or optionally of, 1.9-3.0 years (for Occidental, particularly for American REMBRANDT Neuroglioma Large Database) or 1.9-2.5 years (for Asian, particularly for neuroglioma cases treated by Beijing Tian Tan Hospital in 2006-2009, the Chinese Glioma Genome Atlas, http://jzl.dajiankang.com/portal.php); and

if the ex vivo sample from the neuroglioma patient to be identified has an average expression level of the PM gene group less than that of the EM gene group, the neuroglioma patient to be identified has a survival time prognosis of, or optionally of, 1.3-1.7 years (for Occidental, particularly for American REMBRANDT Neuroglioma Large Database) or 1.1-1.6 years (for Asian, particularly for neuroglioma cases treated by Beijing Tian Tan Hospital in 2006-2009, the Chinese Glioma Genome Atlas, http://jzl.dajiankang.com/portal.php).

In above kit, the average expression level of the PM gene group greater than that of the EM gene group refers to a ratio of the average expression level of the PM gene group to that of the EM gene group of 1.2-5;

the average expression level of the PM gene group equal to that of the EM gene group, without significant difference, refers to a ratio of the average expression level of the PM gene group to that of the EM gene group of 0.9-1.1; and

the average expression level of the PM gene group less than that of the EM gene group refers to a ratio of the average expression level of the PM gene group to that of the EM gene group of 0-0.8, but not 0.

In above kit, the comparison card records following A or B:

A (for American REMBRANDT Neuroglioma Large Database):

the average expression level of the PM gene group greater than that of the EM gene group refers to a ratio of the average expression level of the PM gene group to that of the EM gene group of 1.714±0.032;

the average expression level of the PM gene group equal to that of the EM gene group, without significant difference, refers to a ratio of the average expression level of the PM gene group to that of the EM gene group of 1.074±0.022; and

the average expression level of the PM gene group less than that of the EM gene group refers to a ratio of the average expression level of the PM gene group to that of the EM gene group of 0.592±0.012;

B (for neuroglioma cases treated by Beijing Tian Tan Hospital in 2006-2009, the Chinese Glioma Genome Atlas, http://jzl.dajiankang.com/portal.php):

the average expression level of the PM gene group greater than that of the EM gene group refers to a ratio of the average expression level of the PM gene group to that of the EM gene group of 1.427±0.034;

the average expression level of the PM gene group equal to that of the EM gene group without significant difference refers to a ratio of the average expression level of the PM gene group to that of the EM gene group of 0.939±0.033; and

the average expression level of the PM gene group less than that of the EM gene group refers to a ratio of the average expression level of the PM gene group to that of the EM gene group of 0.460±0.031.

In above kit, the agent comprises a chip that is capable of hybridization with 68 genes in the ex vivo sample or mRNAs or cRNA thereof; and

the chip is particularly a chip of whole human genome oligonucleotide microarray.

cRNAs of the genes described above are prepared by a method comprising: total RNA is extracted from an ex vivo sample, and subjected amplification into cRNAs with an Agilent RNA linear amplification kit (Agilent Low RNA Input Linear Amplification Kit PLUS) and labeled with Cy3, to obtain cRNAs of all genes.

In above kit, the ex vivo sample is an ex vivo tissue of neuroglioma.

The present invention has another goal to provide a set of gene groups for predicting or for auxiliary prediction of prognosis survival time of patients with neuroglioma.

The present invention provides a set of gene groups consisting of a PM gene group and an EM gene group with a total of 68 genes:

the PM gene group consisting of following 39 genes:

C10orf18, C1QL1, C1orf106, C9orf140, CACNG4, CHD7, CSNK1E, EIF4EBP2, ETV1, FAM5C, KLRC3, LIX1L, LOC283174, LPHN3, LPPR1, MARCKS, MEX3A, MMP16, MYT1, NAV1, NLGN1, NOVA1, NXPH1, OLIG1, OLIG2, PATZ1, PCGF2, PDGFRA, POLR2F, RFX7, SOX4, SOX6, SOX8, TACC2, TMCC1, TSHZ1, ZEB1, ZNF22, ZNF462; and

the EM gene group consisting of following 29 genes:

### ACSS3, CDKN2C, DENND2A, DMRTA2, EGFR, ELOVL2, HS3ST3B1, ITGB8, LFNG, NCOA3, NES, NFIA, PDGFA, PMS2P11, POU3F2, PRPF31, RNF180, SALL1, SEC61G, SEMA6D, SHOX2, SNX5, SOCS2, SOX9, TNFRSF19, TRIOBP, UHRF1, VAV3, ZNF558.

Also provided is a use of the kit or the set of gene groups described above in the manufacturer of a product for predicting or for auxiliary prediction of survival time prognosis of a patient with neuroglioma.

Also provided is a use of the set of gene groups described above as a marker in the manufacture of a product for predicting or for auxiliary prediction of survival time prognosis of a patient with neuroglioma.

In above, an average expression level of a gene group refers to an average expression level of mRNAs of the genes in the gene group.

In above, an average expression level of a PM gene group refers to a sum of expression levels of the genes in the PM gene group divided by 39;

In above, an average expression level of an EM gene group refers to a sum of expression levels of the genes in the EM gene group divided by 29.

In above, an expression level of a gene refers to an amount of mRNA expressed from each gene.

The present invention utilizes mRNA expression profile data from neuroglioma databases, to screen out two gene groups (called PM and EM gene groups, respectively) co-expressed with PDGFRA and EGFR, with a total of 68 subtype marking genes. The mRNA levels of multiple or all genes in the PM and EM gene groups are detected by, for example, a gene chip, molecular hybridization, RT-PCR, and the like, allowing for robust diagnosis for subtype identification of a neuroglioma sample and an effective prediction for survival time prognosis of a patient.

The present invention may also use 68 subtype marking genes to make a gene chip for neuroglioma subtype identification, wherein the gene chip is such that cDNAs or oligonucleotide probes of these genes are immobilized into a microarray, in which each of the probes can specifically hybridize with cDNA, mRNA of a corresponding gene, or an amplified product thereof, from a tissue sample. The microarray may be a plate, microbead, needle, or membrane array, and may be an oligonucleotide, polynucleotide, or cDNA array.

The present invention enables neuroglioma subtype identification by detecting expression levels of mRNAs of 68 subtype marking genes in a tissue sample, for example, which may be performed by detecting the expression levels of mRNAs with nucleic acid molecule hybridization probes or RT-PCR amplification. Nucleic acid molecule hybridization may be an in situ hybridization in a tissue section, and RT-PCR may be a quantitative, hemi-quantitative, or qualitative method.

The present invention provides a method for identifying a neuroglioma subtype using 68 subtype marking genes, particularly comprising:

a) extracting total RNA from a carcinoma tissue sample surgically removed from a patient with neuroglioma;

b) amplifying mRNAs in the total RNA of the sample into antisense RNAs (cRNAs) along with fluorescence labeling;

c) hybridizing the labeled sample cRNA with a gene chip (which may be either a gene chip made of 68 subtype marking genes, or a chip of whole human genome oligonucleotide microarray);

d) analyzing the gene expression of the sample with NMF clustering analysis; and calculating a ratio of average expression level of mRNA of a PM gene group to that of an EM gene group in the glioma sample, and comparing it with the ratios of various subtypes, to precisely identify the subtype of neuroglioma, and judging the survival time prognosis of the patient in accordance with the survival time of a corresponding subtype patient.

### DESCRIPTION OF DRAWINGS

FIG. 1 is a PM/EM subtyping gene expression profile of a neuroglioma sample from the REMBRANDT database.

FIG. 2 is analysis of survival time of a molecular subtype of neuroglioma from the REMBRANDT database.

FIG. 3 is a PM/EM subtyping gene expression profile of a neuroglioma sample from the Tian Tan database.

FIG. 4 is analysis of survival time of a molecular subtype of neuroglioma from the Tian Tan database.

### SPECIFIC EMBODIMENTS

All of the experimental methods used in following Examples are conventional methods unless otherwise indicated.

All of the materials, agents, etc. used in following Examples are commercially available unless otherwise indicated.

### EXAMPLE 1

### Screening of Neuroglioma Subtype Marking Gene Group and Determination of Subtyping Criteria

1. Screening of Neuroglioma Subtype Marking Gene Group and Determination of Subtyping Criteria

From the neuroglioma gene expression profile database GSE4290 published by NCBI (http://www.ncbi.nlm.nih.gov/geo/query/acc.cgi?acc=GSE4290&submit.x=0&submit.y=0), a gene group consisting of 37 genes (referred to as EM genes) co-expressed with epidermal growth factor receptor (EGFR), and a gene group consisting of 44 genes (referred to as PM genes) co-expressed with platelet-derived growth factor receptor A (PDGFRA) were obtained through Pearson correlation analysis. These two gene groups were independent of each other, with no overlapping. From unsupervised hierarchical clustering analysis, it was found that the PM gene group and the EM gene group of the glioma sample from the database were expressed in three specific modes: high expression of PM genes and low expression of EM genes (referred to as PM^{high}), low expression of PM genes and high expression of EM genes (referred to as EM^{high}), and low expressions of both genes (referred to as PM^{low}EM^{low}). Thus, using the PM/EM gene group, the neuroglioma sample from the database may be classified, which is referred to as PM/EM subtyping. After removal of part of genes that were not differentially expressed by further filtration screening, 29 EM genes and 39 PM genes, totally 68 genes, were finally identified, constituting a marking gene group for PM/EM subtyping (see Table 1). Then, based on an average expression level of mRNAs of the 39 PM genes (i.e., an average expression level of the PM gene group) relative to an average expression level of the 29 EM genes (i.e., an average expression level of the EM gene group), a PM/EM subtyping criteria was defined as below:

1) if the average expression level of the PM gene group was greater than that of the EM gene group, the sample had a subtype of PM^{high};

2) if the average expression level of the PM gene group was equal to that of the EM gene group, the sample had a subtype of EM^{low}PM^{low}; and

3) if the average expression level of the PM gene group was less than that of the EM gene group, the sample had a subtype of EM^{high}.

The PM/EM subtyping criteria may also be identified particularly based on a ratio of the average expression level of the PM gene group to that of the EM gene group (abbreviated as a PM/EM ratio) in the neuroglioma sample:

1) if the PM/EM ratio=1.2-5, the sample had a subtype of PM^{high};

2) if the PM/EM ratio=0.9-1.1, the sample had a subtype of EM^{low}PM^{low}; and

3) if the PM/EM ratio=0-0.8, the sample had a subtype of EM^{high}, but not 0.

TABLE 1
List of genes of neuroglioma subtype marking gene group (totally 68 genes)

| | | Gene | | |
|---|---|---|---|---|
| | | Accession | | Gene |
| No. | Gene Name | Number | Description of Gene Function | Group |
| 1 | ACSS3 | NM_024560 | acyl-CoA synthetase short-chain | EM |
| | | | family member 3 | |
| 2 | C10orf18 | NM_017782 | chromosome 10 open reading frame | PM |
| | | | 18 | |
| 3 | C1QL1 | NM_006688 | EGF containing fibulin-like | PM |
| | | | extracellular matrix protein 1 | |
| 4 | C1orf106 | NM_018265 | chromosome 1 open reading frame | PM |
| | | | 106 | |
| 5 | C9orf140 | NM_178448 | chromosome 9 open reading frame | PM |
| | | | 140 | |
| 6 | CACNG4 | NM_014405 | calcium channel, voltage-dependent, | PM |
| | | | gamma subunit 4 | |
| 7 | CDKN2C | NM_001262 | cyclin-dependent kinase inhibitor 2C | EM |
| | | | (p18, inhibits CDK4) | |
| 8 | CHD7 | NM_017780 | chromodomain helicase DNA binding | PM |
| | | | protein 7 | |
| 9 | CSNK1E | NM_152221 | casein kinase 1, epsilon | PM |
| 10 | DENND2A | NM_015689 | DENN/MADD domain containing 2A | EM |
| 11 | DMRTA2 | NM_032110 | DMRT-like family A2 | EM |
| 12 | EGFR | NM_005228 | epidermal growth factor receptor | EM |
| 13 | EIF4EBP2 | NM_004096 | eukaryotic translation initiation factor | PM |
| | | | 4E binding protein 2 | |
| 14 | ELOVL2 | NM_017770 | elongation of very long chain fatty | EM |
| | | | acids (FEN1/Elo2, SUR4/Elo3, | |
| | | | yeast)-like 2 | |
| 15 | ETV1 | NM_001163151 | ets variant 1 | PM |
| 16 | FAM5C | NM_199051 | family with sequence similarity 5, | PM |
| | | | member C | |
| 17 | HS3ST3B1 | NM_006041 | heparan sulfate (glucosamine) | EM |
| | | | 3-O-sulfotransferase 3B1 | |
| 18 | ITGB8 | NM_002214 | integrin, beta 8 | EM |
| 19 | KLRC3 | NM_002261 | killer cell lectin-like receptor subfamily | PM |
| | | | C, member 3 | |
| 20 | LFNG | NG_008109 | LFNG O-fucosylpeptide | EM |
| | | | 3-beta-N-acetylglucosaminyltransferase | |
| 21 | LIX1L | NM_153713 | Lix1 homolog (mouse)-like | PM |
| 22 | LOC283174 | NM_024344 | hypothetical LOC283174 | PM |
| 23 | LPHN3 | NM_015236 | latrophilin 3 | PM |
| 24 | LPPR1 | NM_207299 | lipid phosphate phosphatase-related | PM |
| | | | protein type 1 | |
| 25 | MARCKS | NM_002356 | myristoylated alanine-rich protein | PM |
| | | | kinase C substrate | |
| 26 | MEX3A | NM_001093725 | mex-3 homolog A (C. elegans) | PM |
| 27 | MMP16 | NM_005941 | matrix metallopeptidase 16 | PM |
| | | | (membrane-inserted) | |
| 28 | MYT1 | NM_004535 | myelin transcription factor 1 | PM |
| 29 | NAV1 | NM_001167738 | neuron navigator 1 | PM |
| 30 | NCOA3 | NM_181659 | nuclear receptor coactivator 3 | EM |
| 31 | NES | NM_006617 | nestin | EM |
| 32 | NFIA | NM_001145511 | nuclear factor I/A | EM |
| 33 | NLGN1 | NM_014932 | neuroligin 1 | PM |
| 34 | NOVA1 | NM_006491 | neuro-oncological ventral antigen 1 | PM |
| 35 | NXPH1 | NM_152745 | neurexophilin 1 | PM |
| 36 | OLIG1 | NM_138983 | oligodendrocyte transcription factor 1 | PM |
| 37 | OLIG2 | NM_005806 | oligodendrocyte lineage transcription | PM |
| | | | factor 2 | |
| 38 | PATZ1 | NM_032052 | POZ (BTB) and AT hook containing | PM |
| | | | zinc finger 1 | |
| 39 | PCGF2 | NM_007144 | polycomb group ring finger 2 | PM |
| 40 | PDGFA | NM_033023 | platelet-derived growth factor alpha | EM |
| | | | polypeptide | |
| 41 | PDGFRA | NM_006206 | platelet-derived growth factor receptor, | PM |
| | | | alpha polypeptide | |
| 42 | PMS2P11 | NR_023383 | postmeiotic segregation increased 2 | EM |
| | | | pseudogene 11 | |
| 43 | POLR2F | NM_021974 | polymerase (RNA) II (DNA directed) | PM |
| | | | polypeptide F | |
| 44 | POU3F2 | NM_005604 | POU class 3 homeobox 2 | EM |
| 45 | PRPF31 | NM_015629 | PRP31 pre-mRNA processing factor | EM |
| | | | 31 homolog (S. cerevisiae) | |
| 46 | RFX7 | NM_022841 | regulatory factor X, 7 | PM |
| 47 | RNF180 | NM_001113561 | ring finger protein 180 | EM |
| 48 | SALL1 | NG_007990 | sal-like 1 (Drosophila) | EM |
| 49 | SEC61G | NM_014302 | Sec61 gamma subunit | EM |
| 50 | SEMA6D | NM_001198999 | sema domain, transmembrane domain | EM |
| | | | (TM), and cytoplasmic domain, | |
| | | | (semaphorin) 6D | |
| 51 | SHOX2 | NM_003030 | short stature homeobox 2 | EM |
| 52 | SNX5 | NM_152227 | sorting nexin 5 | EM |
| 53 | SOCS2 | NM_003877 | suppressor of cytokine signaling 2 | EM |
| 54 | SOX4 | NM_003107 | SRY (sex determining region Y)-box 4 | PM |
| 55 | SOX6 | NM_033326 | SRY (sex determining region Y)-box 6 | PM |
| 56 | SOX8 | NM_014587 | SRY (sex determining region Y)-box 8 | PM |
| 57 | SOX9 | NM_000346 | SRY (sex determining region Y)-box 9 | EM |
| 58 | TACC2 | NM_206860 | transforming, acidic coiled-coil | PM |
| | | | containing protein 2 | |
| 59 | TMCC1 | NM_001017395 | transmembrane and coiled-coil | PM |
| | | | domain family 1 | |
| 60 | TNFRSF19 | NM_148957 | tumor necrosis factor receptor | EM |
| | | | superfamily, member 19 | |
| 61 | TRIOBP | NM_001039141 | TRIO and F-actin binding protein | EM |
| 62 | TSHZ1 | NM_005786 | teashirt zinc finger homeobox 1 | PM |
| 63 | UHRF1 | NM_001048201 | ubiquitin-like with PHD and ring finger | EM |
| | | | domains 1 | |
| 64 | VAV3 | NM_006113 | vav 3 guanine nucleotide exchange | EM |
| | | | factor | |
| 65 | ZEB1 | NM_001128128 | zinc finger E-box binding homeobox 1 | PM |
| 66 | ZNF22 | NM_006963 | zinc finger protein 22 (KOX 15) | PM |
| 67 | ZNF462 | NM_021224 | zinc finger protein 462 | PM |
| 68 | ZNF558 | NM_144693 | zinc finger protein 558 | EM |

2. Verification of Neuroglioma PM/EM Subtyping Criteria and Analysis of Survival Time Prognosis

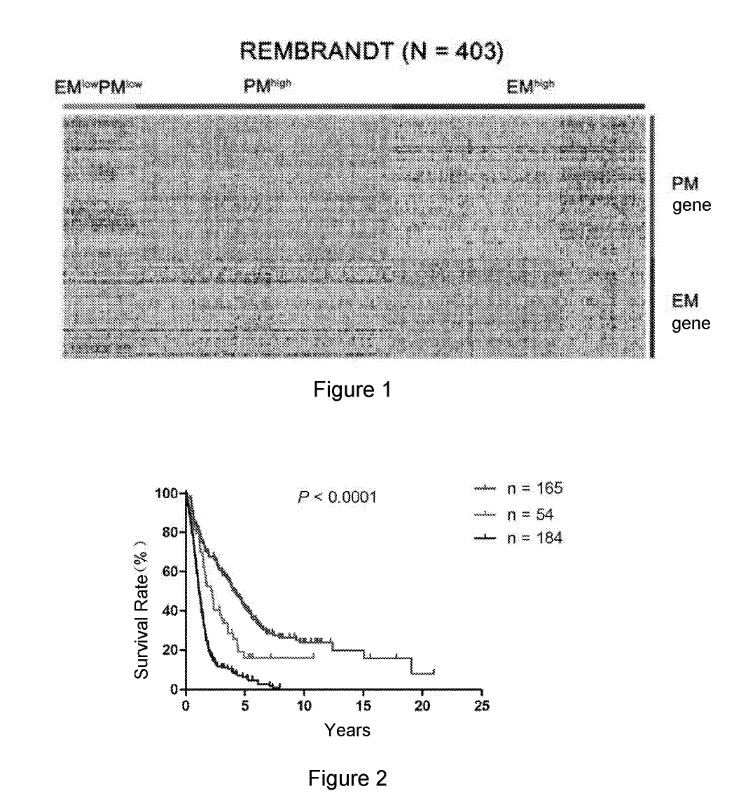

Taking use of mRNA expression profile data of 403 neuroglioma samples with survival time prognosis information from the American REMBRANDT Neuroglioma Large Database (a public data platform established by cooperation of several medical research institutes in U.S., http://caintegrator-info.nci.nih.gov/rembrandt), above PM/EM subtyping criteria was verified, and the relation of a PM/EM subtype to survival time prognosis was examined. Based on the glioma classification criteria from WHO, the 403 neuroglioma samples were classified into pathologically diagnosed types including 109 Astrocytoma (Grade II, III), 193 Glioblastoma (Glioblastoma Multiforme (GBM); Grade IV), 51 Oligodendroglioma (Grade II, III), 7 Oligoastrocytoma (Grade II, III), and 43 undefinitely diagnosed gliomas. From the mRNA expression profiles for PM and EM gene groups, unsupervised clustering analysis was conducted using Nonnegative Matrix Factorization (NMF) clustering algorithm, like GSE4290 database, the neuroglioma samples from the REMBRANDT database were presented three subtypes (FIG. 1):

1) a subtype of PM^{high}, 165 cases, with an average expression level of the PM gene group greater than that of the EM gene group;

2) a subtype of EM^{high}, 184 cases, with an average expression level of the PM gene group equal to that of the EM gene group; and

3) a subtype of EM^{low}PM^{low}, 54 cases, with an average expression level of the PM gene group less than that of the EM gene group.

All of the pathologically diagnosed types were covered by each of the PM/EM subtypes, and the glioma samples with undefined morphological diagnosis were also explicitly classified into one of the subtypes of PM^{high}, EM^{low}PM^{low}, and EM^{high}, respectively. The ratio of the average expression level of the PM gene group to that of the EM gene group (i.e., the PM/EM ratio) in the glioma sample of each subtype fell within the ratio range of the corresponding subtype defined as PM/EM subtyping criteria described above in section 1, and there were very significant differences between the three subtypes as shown in a statistical test (Table 2, p<0.0001, Kruskal-Wallistest).

The average expression level of the gene group refers to an average of expression levels of mRNAs of all genes in the gene group.

TABLE 2
PM/EM subtypes of a neuroglioma sample from the
REMBRANT database

| | PM^{high} | EM^{low}PM^{low} | EM^{high} |
|---|---|---|---|
| PM/EM ratio | 1.714 ± 0.032 | 1.074 ± 0.022 | 0.592 ± 0.012 |
| (Mean ± SEM) | | | |

It was found by analysis of survival time that patients with different subtypes, i.e., PM^{high} (165 patients), EM^{low}PM^{low} (54 patients) and EM^{high} (184 patients), had significantly difference in prognosis (FIG. 2, p<0.0001, Log-rank test). Patients with PM^{high} and EM^{low}PM^{low} showed better prognosis, 95% of the patients having survival time prognosis of 3.7-4.9 years and 1.9-3.0 years, respectively; and patients with EM^{high} showed poorer prognosis, 95% of the patients having survival time prognosis of 1.3-1.7 years.

It is indicated from above results that the PM/EM subtyping may be used for predicting survival time of a patient with glioma:

If the average expression level of the PM gene group is greater than that of the EM gene group (that is, the sample has a molecular subtype of PM^{high}) or equal to that of the EM gene group (that is, the sample has a molecular subtype of EM^{low}PM^{low}), the sample is assigned a survival time prognosis of over or optionally over 1.9 years (1.9 years inclusive); wherein, if sample is of a molecular subtype of PM^{high}; particularly, the survival time prognosis thereof is or optionally is 3.7-4.9 years; if the sample is of a molecular subtype of EM^{low}PM^{low}; particularly, the survival time prognosis thereof is or optionally is 1.9-3.0 years;

If the average expression level of the PM gene group is less than that of the EM gene group (that is, the sample has a molecular subtype of EM^{high}), the sample is assigned a survival time prognosis of no more than or optionally no more than 1.9 years (1.9 years exclusive); and particularly, the survival time prognosis of this molecular subtype is or optionally is 1.3-1.7 years.

Specifically, the subtyping criteria of a neuroglioma sample to be identified may also be further determined from a ratio of the average expression level of the PM gene group to that of the EM gene group of the neuroglioma sample, and the survival time of each of patients with respective subtypes may be predicted:

if the PM/EM ratio=1.2-5, the sample has a molecular subtype of PM^{high}; with a survival time prognosis of, or optionally of, 3.7-4.9 years;

if the PM/EM ratio=0.9-1.1, the sample has a molecular subtype of EM^{low}PM^{low}; with a survival time prognosis of, or optionally of, 1.9-3.0 years; and

if the PM/EM ratio=0-0.8, but not 0; the sample has a molecular subtype of EM^{high}; with a survival time prognosis of, or optionally of, 1.3-1.7 years.

For the neuroglioma samples from the REMBRANT database, above criteria may be particularly as below:

if the PM/EM ratio=1.714±0.032, the sample has a molecular subtype of PM^{high}; with a survival time prognosis of, or optionally of, 3.7-4.9 years;

if the PM/EM ratio=1.074±0.022, the sample has a molecular subtype of EM^{low}PM^{low}; with a survival time prognosis of, or optionally of, 1.9-3.0 years; and

if the PM/EM ratio=0.592±0.012; the sample has a molecular subtype of EM^{high}; with a survival time prognosis of, or optionally of, 1.3-1.7 years.

### EXAMPLE 2

### Use of Neuroglioma Subtype Marking Gene Group and Subtyping Criteria in Prediction of Survival Time Prognosis for Neuroglioma Patient to be Identified

I. Detection of Gene Expression Level

1. Selection of Patients and Treatment of Samples

209 samples were from neuroglioma patients treated by the Beijing Tian Tan Hospital in 2006-2009 (patients information; the Chinese Glioma Genome Atlas, http://jzl.dajiankang.com/portal.php), which, based on the glioma classification criteria from WHO, were classified into pathological types including Astrocytoma (Grade II, 58 cases; and Grade III, 8 cases), Glioblastoma (Astrocytoma Grade IV, GBM, 79 cases), Oligodendroglioma (Grade II, 18 cases; and Grade III, 11 cases), Oligoastrocytoma (Grade II, 20 cases; and Grade III, 15 cases).

Glioma tissue samples were confirmed via pathological diagnosis, and quick-frozen by liquid nitrogen, stored at −80° C.

2. Hybridization with Gene Chip

Total RNA was extracted from each of the glioma tissue samples using a total RNA isolation kit (AM1830; Ambion, Austin, Tex.), and measured for concentration by NanoDropND-1000 spectrophotometer (NanoDropND Technologies, Houston, Tex.).

mRNAs were amplified, with an Agilent RNA linear amplification kit (Agilent Low RNA Input Linear Amplification Kit PLUS), into cRNAs, and Cy3-labeled.

The fluorescence labeled cRNA products were hybridized with a chip of Agilent Whole Human Genome Oligo Microarray of 4×44 (G4845A; Agilent Technologies). Then, the hybridized chip was washed, and subjected to image scanning through an Agilent G2565 BA gene chip microarray scanning system. The labeling, hybridization, washing and scanning of all samples strictly followed the Operation instructions from the manufacturer of the gene chip.

The fluorescence intensity of the image was read and pre-treated using Agilent Feature Extraction Software (v9.1) (Agilent Feature Extraction Software). The GeneSpring GX 11.0 (Agilent Technologies) was used to achieve data normalization and screening. Only the genes labeled as present or higher than a lower marginal could pass the quality filtration screening. The data of the gene chip was normalized to 50% of the fluorescence intensity of the chip.

II. Sample Subtyping

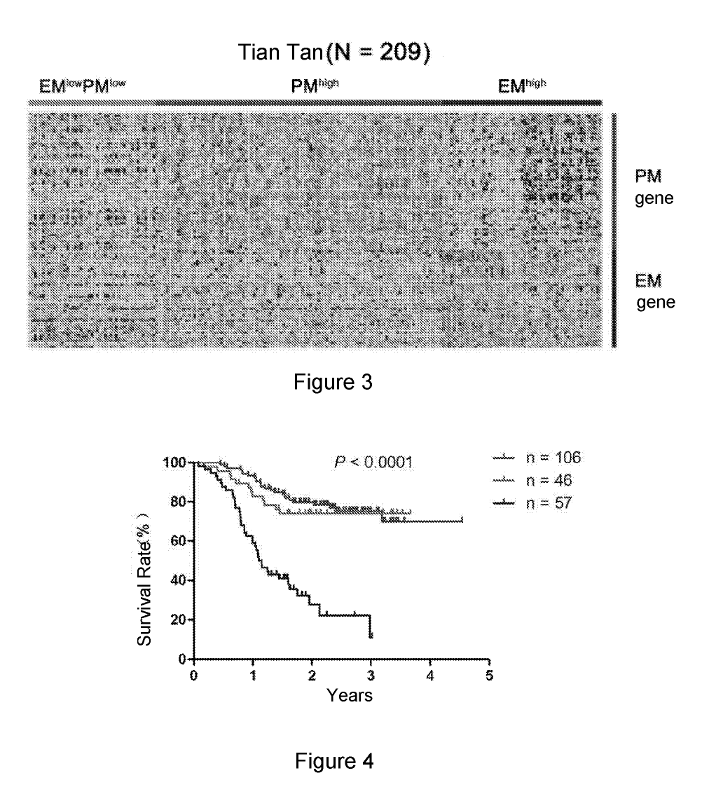

Based on the expression levels of respective genes represented by the values of the fluorescence intensity thereof, unsupervised clustering analysis was conducted using Nonnegative Matrix Factorization (NMF) clustering, to obtain PM/EM gene expression profiles for explicit classification of neuroglioma samples, as shown in FIG. 3, which is consistent with above two databases. The glioma samples from the Tian Tan database were divided into three specific types: 1) a subtype of PM^{high}, 106 patients, with an average expression level of PM gene group greater than that of EM gene group; 2) a subtype of EM^{low}PM^{low}, 46 patients, with the average PM gene group expression level equal to the average EM gene group expression level; and 3) a subtype of EM^{high}, 57 patients, with the average PM gene group expression level less than the average EM gene group expression level.

Ratios of the average expression level of PM gene group to that of the EM gene group (abbreviated as a PM/EM ratio) was calculated for the glioma samples of various subtypes, and the patients with the subtypes was analyzed for survival times.

The average expression level of a gene group refers to an average of mRNA expression levels of all genes in the gene group.

The results are shown in FIG. 3 and Table 3, and FIG. 3 shows the PM/EM gene expression profile of 209 neuroglioma samples, which may be definitely divided into three subtypes: PM_{high} (106 patients), EM^{low}PM^{low} (46 patients), and EM^{high} (57 patients). It was shown by statistical analysis that each of the subtypes had an average expression level ratio of the PM gene group to the EM gene group (abbreviated as a PM/EM ratio) falling within the ratio range of a corresponding subtype defined above in section 1. These subtypes are significantly different to each other (p<0.0001, Kruskal-Wallistest; Table 3).

TABLE 3
PM/EM subtypes of neuroglioma samples from the Tian Tan database

| | PM^{high} | EM^{low}PM^{low} | EM^{high} |
|---|---|---|---|
| PM/EM Ratio | 1.427 ± 0.034 | 0.939 ± 0.033 | 0.460 ± 0.031 |
| (Mean ± SEM) | | | |

Survival time analysis in December, 2012 showed that patients with different subtypes, i.e., PM^{high} (106), EM^{low}PM^{low} (46) and EM^{high} (57) had significant different prognosis (FIG. 4, p<0.0001, Log-rank test). PM^{high} patients and EM^{low}PM^{low} patients had better prognosis, 95% of the patients have survival time prognosis of 2.3-2.7 years and 1.9-2.5 years, respectively; and EM^{high} patients had poorer prognosis, 95% of the patients have survival time prognosis of 1.1-1.6 years.

It is indicated from above results that PM/EM subtyping may be used for predicting the survival time of a patient with glioma:

if the average expression level of the PM gene group is greater than that of the EM gene group (the sample has a molecular subtype of PM^{high}) or equal to that of the EM gene group (the sample has a molecular subtype of EM^{low}PM^{low}), the sample has a survival time prognosis of, or optionally of, over 1.9 years (1.9 years inclusive); wherein, if the sample has a molecular subtype of PM^{high}; the survival time prognosis thereof is or optionally is 2.3-2.7 years; if the sample has a molecular subtype of EM^{low}PM^{low}; particularly, the survival time prognosis thereof is or optionally is 1.9-2.5 years;

if average expression level of the PM gene group is less than that of the EM gene group (the sample has a molecular subtype of EM^{high}), the sample has a survival time prognosis of, or optionally of, no more than 1.9 years (1.9 years exclusive); particularly, the survival time prognosis thereof is or optionally is 1.1-1.6 years.

Specifically, the type of a neuroglioma sample to be identified may also be identified from a ratio of the average expression level of the PM gene group to that of the EM gene group in the neuroglioma sample, and the patients with various subtypes may be predicted with a survival time:

if the PM/EM ratio=1.2-5, the sample has a molecular subtype of PM^{high}; with a survival time prognosis of, or optionally of, 2.3-2.7 years;

if the PM/EM ratio=0.9-1.1, the sample has a molecular subtype of EM^{low}PM^{low}; with a survival time prognosis of, or optionally of, 1.9-2.5 years; and

if the PM/EM ratio=0-0.8, but not 0; the sample has a molecular subtype of EM^{high}; with a survival time prognosis of, or optionally of, 1.1-1.6 years.

For the neuroglioma patients treated by the Beijing Tian Tan Hospital, above criteria may also be particularly as below:

if the PM/EM ratio=1.427±0.034, the sample has a molecular subtype of PM^{high}; with a survival time prognosis of, or optionally of, 2.3-2.7 years;

if the PM/EM ratio=0.939±0.033, the sample has a molecular subtype of EM^{low}PM^{low}; with a survival time prognosis of, or optionally of, 1.9-2.5 years; and

if the PM/EM ratio=0.460±0.031; the sample has a molecular subtype of EM^{high}; with a survival time prognosis of, or optionally of, 1.1-1.6 years.

As can be seen from above experiments, the expression profile data of the PM/EM gene group enables precise molecular subtype diagnosis of a glioma sample. Such subtyping can be effective in judging the prognosis of a patient, and contributive to find a therapy target.

### INDUSTRIAL APPLICATION

In the present invention, the experiments demonstrate that the subtype marking gene groups obtained by the present invention, consisting of two major gene groups co-expressed with PDGFRA and EGFR with a total of 68 genes, can stably divide neuroglioma samples from different database sources into three specific subtypes, which can significantly overcome the restriction in the prior morphological diagnosis, and may be used for performing clinical diagnosis and directing clinical therapy of neuroglioma, as well as for judging the survival time prognosis of a neuroglioma patient in a relatively precise way. Moreover, since the 68 genes are closely associated with the proliferation and differentiation of neural stem cells and progenitor cells as well as development and progression of tumors, the present invention provides an important guide for revealing cytologic and genetic origins of neuroglioma, and in turns for finding and screening a therapy target; and a rapid and convenient detection platform for establishing a model in relation to screening a therapeutic compound, and the like. Therefore, the present invention can be widely used in scientific research, medical, pharmacy and other fields.

1. A kit for predicting or for auxiliary prediction of survival time prognosis of a patient with neuroglioma, comprising an agent for measuring the expression level of each of genes of a PM gene group and an EM gene group in ex vivo sample from a neuroglioma patient to be identified, and a comparison card;
the PM gene group consisting of following 39 genes:
C10orf18, C1QL1, C1orf106, C9orf140, CACNG4, CHD7, CSNK1E, EIF4EBP2, ETV1, FAM5C, KLRC3, LIX1L, LOC283174, LPHN3, LPPR1, MARCKS, MEX3A, MMP16, MYT1, NAV1, NLGN1, NOVA1, NXPH1, OLIG1, OLIG2, PATZ1, PCGF2, PDGFRA, POLR2F, RFX7, SOX4, SOX6, SOX8, TACC2, TMCC1, TSHZ1, ZEB1, ZNF22, ZNF462;
the EM gene group consisting of following 29 genes:
ACSS3, CDKN2C, DENND2A, DMRTA2, EGFR, ELOVL2, HS3ST3B1, ITGB8, LFNG, NCOA3, NES, NFIA, PDGFA, PMS2P11, POU3F2, PRPF31, RNF180, SALL1, SEC61G, SEMA6D, SHOX2, SNX5, SOCS2, SOX9, TNFRSF19, TRIOBP, UHRF1, VAV3, ZNF558; and
the comparison card recording that:
if the ex vivo sample from the neuroglioma patient to be identified has an average expression level of the PM gene group greater than or equal to that of the EM gene group, the neuroglioma patient to be identified has a survival time prognosis over or optionally over 1.9 years; and
if the ex vivo sample from the neuroglioma patient to be identified has an average expression level of the PM gene group less than that of the EM gene group, the neuroglioma patient to be identified has a survival time prognosis of no more than or optionally no more than 1.9 years.

2. The kit according to claim 1, wherein:
the comparison card records that:
if the ex vivo sample from the neuroglioma patient to be identified has an average expression level of the PM gene group greater than that of the EM gene group, the neuroglioma patient to be identified has a survival time prognosis of, or optionally of, 3.7-4.9 years or 2.3-2.7 years;
if the average expression level of the PM gene group is equal to that of the EM gene group, the neuroglioma patient to be identified has a survival time prognosis of, or optionally of, 1.9-3.0 years or 1.9-2.5 years; and
if the ex vivo sample from a neuroglioma patient to be identified has an average expression level of the PM gene group less than that of the EM gene group, the neuroglioma patient to be identified has a survival time prognosis of, or optionally of, 1.3-1.7 years or 1.1-1.6 years.

3. The kit according to claim 1, wherein:
the average expression level of the PM gene group greater than that of the EM gene group refers to a ratio of the average expression level of the PM gene group to that of the EM gene group of 1.2-5;
the average expression level of the PM gene group equal to that of the EM gene group without significant difference refers to a ratio of the average expression level of the PM gene group to that of the EM gene group of 0.9-1.1; and
the average expression level of the PM gene group less than that of the EM gene group refers to a ratio of the average expression level of the PM gene group to that of the EM gene group of 0-0.8, but not 0.

4. The kit according to claim 1, wherein:
the comparison card records following A or B:
A:
the average expression level of the PM gene group greater than that of the EM gene group refers to a ratio of the average expression level of the PM gene group to that of the EM gene group of 1.714±0.032;
the average expression level of the PM gene group equal to that of the EM gene group without significant difference refers to a ratio of the average expression level of the PM gene group to that of the EM gene group of 1.074±0.022; and
the average expression level of the PM gene group less than that of the EM gene group refers to a ratio of the average expression level of the PM gene group to that of the EM gene group of 0.592±0.012;
B:
the average expression level of the PM gene group greater than that of the EM gene group refers to a ratio of the average expression level of the PM gene group to that of the EM gene group of 1.427±0.034;
the average expression level of the PM gene group equal to that of the EM gene group without significant difference refers to a ratio of the average expression level of the PM gene group to that of the EM gene group of 0.939±0.033; and
the average expression level of the PM gene group less than that of the EM gene group refers to a ratio of the average expression level of the PM gene group to that of the EM gene group of 0.460±0.031.

5. The kit according to claim 1, wherein:
the agent comprises a chip that is capable of hybridization with 68 genes in the ex vivo sample, or mRNAs or cRNAs thereof; and
the chip is particularly a chip of whole human gene genome oligonucleotide microarray.

6. The kit according to claim 1, wherein:
the ex vivo sample is an ex vivo tissue of neuroglioma.

7. A set of gene groups for predicting or for auxiliary prediction of survival time prognosis of a patient with neuroglioma, consisting of a PM gene group and an EM gene group, a total of 68 genes:
the PM gene group consisting of following 39 genes:
C10orf18, C1QL1, C1orf106, C9orf140, CACNG4, CHD7, CSNK1E, EIF4EBP2, ETV1, FAM5C, KLRC3, LIX1L, LOC283174, LPHN3, LPPR1, MARCKS, MEX3A, MMP16, MYT1, NAV1, NLGN1, NOVA1, NXPH1, OLIG1, OLIG2, PATZ1, PCGF2, PDGFRA, POLR2F, RFX7, SOX4, SOX6, SOX8, TACC2, TMCC1, TSHZ1, ZEB1, ZNF22, ZNF462; and
the EM gene group consisting of following 29 genes:
ACSS3, CDKN2C, DENND2A, DMRTA2, EGFR, ELOVL2, HS3ST3B1, ITGB8, LFNG, NCOA3, NES, NFIA, PDGFA, PMS2P11, POU3F2, PRPF31, RNF180, SALL1, SEC61G, SEMA6D, SHOX2, SNX5, SOCS2, SOX9, TNFRSF19, TRIOBP, UHRF1, VAV3, ZNF558.

8. (canceled)

9. (canceled)
